(19) United States
(12) Patent Application Publication
(10) Pub. No.: US 2002/0034549 A1
(43) Pub. Date: Mar. 21, 2002

(54) MODIFIED STARCH COATING

(76) Inventors: NATHANIEL T. BECKER, BURLINGAME, CA (US); CHRISTENSEN I. ROBERT JR., PINOLE, CA (US); GEBERT S. MARK, SOUTH SAN FRANCISCO, CA (US)

Correspondence Address: KRISTEN A ANDERSON, GENENCOR INTERNATIONAL INC, 925 PAGE MILL ROAD, PALO ALTO, CA, 943041013

(21) Appl. No.: 09/285,632

(22) Filed: Apr. 2, 1999

Related U.S. Application Data
(60) Provisional application No. 60/080,424, filed on Apr. 2, 1998.

(51) Int. Cl. A61K 9/14

(52) U.S. Cl. 424/489

(57) ABSTRACT

Coatings for pharmaceutical dosage forms, food and confectionery tablets, seeds and granule cores are described. The coating includes a modified starch and a plasticizer optionally in combination with a secondary polymer. Also described is a coating including a modified starch and a secondary polymer optionally in combination with a plasticizer. Also described are coated pharmaceutical dosage forms, food and confectionery tablets, seeds and granule cores. Further described are cleaning, textile and feed compositions including the coated granule cores.

### RELATED APPLICATIONS

This application is a continuation-in-part of U.S. Provisional Application No. 60/080,424, filed Apr. 2, 1998, all of which is hereby incorporated herein in its entirety.

### BACKGROUND OF THE INVENTION

Coatings have long been used on seeds, pharmaceutical dosage forms, food or confectionery tablets, and granules such as enzymes granules to impart desirable characteristics to the final coated product. Developing coatings which have desirable properties is an ongoing source of research and development.

Thin film coating of pharmaceutical tablets allows efficient, controlled, uniform and reproducible coats. Use of multiple layers of coating, such as the polymeric undercoat, polymeric pigmented second coat and polymeric finish coat allows the preparation of very smooth glossy tablets (Ohno, U.S. Pat. No. 4,001,390).

Numerous methods for pan-coating pharmaceutical tablets have been developed and are summarized in Pharmaceutical Dosage Forms: Tablets, Volume 3 (eds. Lieberman and Lachman, 1982, Marcel Dekker). They include sugar-coating techniques, solvent film coating, aqueous film coating, delayed release coating, and granule coating. Pulverized medicine may also be wrapped in a transparent, glossy, resistant, soluble or semi-permeable film as provided by Motoyama et al. (U.S. Pat. No. 4,154,636).

Pharmaceutical tablets have been coated for a variety of reasons, including masking objectionable flavors or odors, protecting unstable tablet compositions, providing protection of the tablet through the stomach with enteric coatings, improving the appearance of the tablet or separating medicine ingredients into a core segment and coating segment.

Aspirin tablets or other tablets that are powdery, easily dissolved and friable have been treated with a variety of coatings to keep them from dissolving too soon (John et al., U.S. Pat. No. 4,302,440). Also, other polymers in non-aqueous vehicles have been used to granulate tablets (Gans et al., U.S. Pat. No. 3,388,041) or to coat onto tablets (Jeffries, U.S. Pat. No. 3,149,040) to protect from dissolving in the stomach or to delay the drug's release. Other non-aqueous film-coating systems have been designed to be applied to a variety of tablets containing a variety of active ingredients as illustrated by Singiser, U.S. Pat. No. 3,256,111 and Brindamour, U.S. Pat. No. 3,383,236. The aqueous coating processes are environmentally more safe than the non-aqueous processes, which involve the use of organic solvents in film-coating solutions. Thin film coatings, which do not alter the dissolution characteristics of the tablet, may be readily formed using aqueous film-coating processes. Unless adequately thick or insoluble coatings are used, most coatings are not capable of effectively masking the strong objectionable bitter taste of triprolidine hydrochloride or other compounds with similar properties.

Seed coating is a practice which has become widespread. It is aimed in particular at improving the germination characteristics, at providing various additives capable of intervening at any time during the growth of plants, at protecting the seeds or at imparting to the seed a shape of a size which is suitable for automatic sowing.

Granules such as enzyme-containing granules can also benefit from the presence of a coating. For example, it is desirable to coat enzyme granules in order to provide a cosmetic white or colored appearance, improve particle strength, reduce the tendency to dust in handling, reduce exposure of workers to enzymes and protect the enzyme against inactivation by moisture, oxidants and other harsh compounds. At the same time, it is important that such coatings not interact negatively with other detergent components. A coating material should also be easy to apply to the granule without excessive agglomeration or yield loss, typically by spraying onto the enzyme granules in a fluidized bed or tumbling coater.

### SUMMARY OF THE INVENTION

The present invention provides a coating including a modified starch and a plasticizer. The modified starch is preferably hydroxypropyl modified starch. The plasticizer is preferably glycerol. The coating can further comprise a secondary polymer.

The present invention further provides a coating including a modified starch and a secondary polymer. The modified starch is preferably hydroxypropyl modified starch. The secondary polymer is preferably methyl cellulose. The coating can further comprise a plasticizer.

The present invention also provides a granule including a granule core and the coating of the present invention. Also provided are cleaning compositions, textile compositions and feed compositions including these granules.

The present invention additionally provides a composition including a tablet and the coating of the present invention, a coated pharmaceutical dosage form including a pharmaceutical dosage form and the coating of the present invention, a coated seed including a seed and and the coating of the present invention.

### DETAILED DESCRIPTION OF THE INVENTION

A coating has been developed which provides the above desirable properties without any apparent negative interactions with detergent components. This coating consists of a modified starch in combination with a plasticizer and optionally a secondary polymer such as a modified cellulose. Another coating can be a modified starch in combination with a secondary polymer and optionally a plasticizer.

In general, unmodified starch or cellulose is not a good coating material. For example, generally, starch is not soluble unless gelatinized by cooking at elevated temperatures, and even then it is usually only partially soluble. Further, neither raw nor cooked starch is a good film former, nor is it easily plasticized. Unmodified cellulose is also insoluble in water.

Modified starch on its own is also not, in general, a good coating material and does not have all of the desired properties for a coating. However, it has been found that by adding a plasticizer such as glycerol, the combined modified starch/plasticizer not only has good solubility and barrier properties but is also a good coating material with excellent mechanical properties.

It has also been found that blends of modified starch and a secondary polymer such as modified cellulose have an advantage in that, for example, they combine the superior film-forming properties of modified cellulose, with the greater solubility and barrier properties of modified starch. The mechanical resilience of these films can be further improved by addition of plasticizers. A blend containing equal parts of each of these polymers, preferably with added plasticizers and pigments, has excellent film strength, good moisture barrier characteristics, and it is feasible to coat from a high solids (15-20% w/w) solution. Also, it is not tacky and can be coated onto, for example, granules or tablets without causing agglomeration.

Preferred starches have been modified in order to, for example, improve the solubility of the starch. Modified starches include starches that have been modified, for example, by acid thinning, debranching, cross-linking, instantization via jet cooking and spray drying or instantization via high temperature extrusion. Modifications to the starch include ethylation (ethyl group substitution), acetylation (acetyl group substitution), methylation (methyl group substitution), hydroxy-propyl substitution, hydroxy-ethyl substitution, carboxy-methyl substitution and hydroxypropyl methyl substitution. Examples of modified starches include:

| | |
|---|---|
| Pure Cote (B760 and B 790) | GPC |
| Pure Set 765 | GPC |
| Potato starch T1-T5 | Western Polymer |
| Amiogum 23 | Cerestar (formerly American Maize) |
| Amiogum 30 | Cerestar (formerly American Maize) |
| Amiogum 50 | Cerestar (formerly American Maize) |
| Amerimaize 2217 | Cerestar (formerly American Maize) |
| Amerimaize 2300 | Cerestar (formerly American Maize) |
| Crisp Tex | Cerestar (formerly American Maize) |
| Batter Tex | Cerestar (formerly American Maize) |
| Amylean 1 | Cerestar (formerly American Maize) |
| Ethylex gums (2015, 2035, 2040 | AE Staley |
| and 2065) | |
| Mira-Gel | AE Staley |
| Soft-Set | AE Staley |
| Ultra-Set | National Starch |
| Capsule starch | National Starch |
| Amylogum CLS | Avebe |

Preferred modified starches are those that have hydroxypropyl substitutions. More preferably, the modified starch is Pure Cote.

Preferred plasticizers include fructose, high fructose corn syrup, glucose, lactose, maltose, galactose, raffinose/sucrose mixture, and other mono- and di-saccharide sugars, sugar alcohols such as glycerol and sorbitol, polyethylene glycols (MW 200-8000), nonionic surfactants such as linear alcohol ethoxylates and alkylphenol ethoxylates, polyols such as Neodol 23/6.5 and propylene glycol, maltodextrin, urea, triethylcitrate (TEC), citric acid, and other carboxylic acids or salts thereof.

Preferred secondary polymers include modified celluloses, polyvinyl alcohol (PVA), polyvinyl pyrrolidone (PVP) and polyacrylamide. Modified celluloses include ethylcellulose, methylcellulose, propylcellulose, hydroxypropyl cellulose, cellulose esters and mixed esters such as: cellulose acetate, cellulose acetate butyrate (CAB), and cellulose acetate propionate (CAP).

The coating of the present invention may further comprise one or more of the following: extenders, lubricants, and pigments. Suitable pigments useful in the coating of the present invention include, but are not limited to, finely divided whiteners such as titanium dioxide or calcium carbonate or colored pigments and dyes or a combination thereof. Preferably such pigments are low residue pigments upon dissolution. Suitable extenders include sugars such as sucrose or starch hydrolysates such as maltodextrin and corn syrup solids, clays such as kaolin and bentonite and talc. Suitable lubricants include nonionic surfactants such as Neodol, tallow alcohols, fatty acids, fatty acid salts such as magnesium stearate and fatty acid esters, lecithin and waxes such as carnauba wax and beeswax.

The coating described herein may be applied by methods known to those skilled in the art of enzyme granulation, including pan-coating, fluid-bed coating, spray drying, or combinations of these techniques.

The coating of the present invention can be a final, outer coating or an inner layer such as in the case of a layered granule core.

The coating of the present invention can be used to coat, for example, pharmaceutical dosage forms, confectionery or food tablets, seeds, or granule cores to produce coated pharmaceutical dosage forms, confectionery or food tablets, seeds, or granules.

Pharmaceutical dosage forms that can be coated with the coating of the present invention include tablets, capsules, caplets and geltabs such as medicinal tablets or vitamin tablets. A large number of pharmaceutical dosage forms that can be coated with the coating of the present invention are known to those of skill in the art. Some methods for coating pharmaceutical dosage forms are described in Pharmaceutical Dosage Forms: Tablets, Volume 3 (eds. Lieberman and Lachman, 1982, Marcel Dekker). Similar methods can be used to coat confectionery or food tablets such as non-pareils, chewing gum balls, pieces of candy and the like.

Methods for coating seeds are well known in the art such as those described in U.S. Pat. No. 4,879,839.

Granule cores that can be coated with the coating of the present invention include those made according to the methods described in, for example, U.S. Pat. 5,324,649; U.S. patent application Ser. No. 09/215,095; U.S. patent application Ser. No. 091215,086; or U.S. Pat. No. 4,740,469. The granule cores can be commercially available granules such as Purafect granules (Genencor International Inc., Rochester, N.Y.) or Savinase granules (Novo Nordisk, Denmark).

The coated granule cores or granules can be used in, for example, cleaning compositions, compositions for use in treating textiles or for use in feed or food, e.g., baking.

The granules of the invention are useful in formulating various detergent compositions or personal care formulations such as shampoos or lotions. A number of known compounds are suitable surfactants useful in compositions comprising the granules of the invention. These include nonionic, anionic, cationic or zwitterionic detergents, as disclosed in U.S. Pat. No. 4,404,128 to Barry J. Anderson and U.S. Pat. No. 4,261,868 to Jiri Flora, et al. A suitable detergent formulation is that described in Example 7 of U.S. Pat. No. 5,204,015 (previously incorporated by reference). The art is familiar with the different formulations which can be used as cleaning compositions.

Granules of the invention can be included in known powdered and liquid detergents. The addition of the granules of the invention to conventional cleaning compositions does not create any special use limitation.

The present invention also relates to cleaning compositions containing the granules of the invention. The cleaning compositions may additionally contain additives which are commonly used in cleaning compositions. These can be selected from, but not limited to, bleaches, surfactants, builders, enzymes and bleach catalysts. It would be readily apparent to one of ordinary skill in the art what additives are suitable for inclusion into the compositions. The list provided herein is by no means exhaustive and should be only taken as examples of suitable additives. It will also be readily apparent to one of ordinary skill in the art to only use those additives which are compatible with the enzymes and other components in the composition, for example, surfactant.

When present, the amount of additive present in the cleaning composition is from about 0.01% to about 99.9%, preferably about 1% to about 95%, more preferably about 1% to about 80%.

The granules of the present invention can be included in animal feed as a delivery vehicle for animal feed additives such as those described in, for example, U.S. Pat. Nos. 5,612,055; 5,314,692; and 5,147,642.

One aspect of the invention is a composition for the treatment of a textile that includes granules of the present invention. For example, a cellulase can be incorporated in the granule and used in a process to treat denim as is well known in the art.

The following examples are representative and not intended to be limiting.

### EXAMPLES

### Example 1

Seed: 25% of batch weight Sucrose, sieved

Spray 1: Matrix Layer: 41.33% of Batch Weight

1. Enzyme concentrate to achieve payload

2. Sucrose

3. Corn starch

Sucrose and corn starch were added directly to the UF concentrate at a 55% sucrose: 45% corn starch ratio. After calculating the amount of UF concentrate needed to achieve the desired payload, sucrose and corn starch were added to the matrix solution. The sucrose was added to the UF concentrate and mixed for 10 minutes. The corn starch was added next with moderately vigorous agitation. The corn starch was dispersed after 20-30 minutes. The matrix layer was sprayed on the sucrose seed in a fluidized bed granulator under the conditions noted in Table 1.

Spray 2: 20% of batch weight

MgSO₄.7H₂O

A 50% solution of the MgSO₄.7H₂O (1:1 MgSO₄.7H₂O:water) was sprayed on the granules above in a fluidized bed granulator under the conditions noted in Table 1.

Spray 3

Coating: 3.67% of batch weight

1. 2.5% Methylcellulose A-15

2. 2.5% Pure Cote B790

3. 6% TiO2

4. 1.0 Neodol

5. 1.67% PEG 600

This outer coating was batched as an 18% dry solids solution. Cold water was added to a vessel, then the Pure Cote B790 and TiO2 was added into the cold water. The Pure Cote and TiO2 was agitated for 10-15 minutes to aid in dispersion. After the Pure Cote and TiO2 has had time to disperse the temperature was brought up to 95° C. The temperature was kept at 95° C. for 30 minutes. While the temperature remained at 95° C., the methylcellulose (MC) A-15 was added. Generally, the MC disperses at a temperature above 60° C. After the 30 minutes at 95° C., the solution was cooled down to 20° C. At 30° C., the MC A-15 dissolved. The PEG 600 and Neodol were added at this time. After 30 minutes, this solution was used in the coater. The coating was sprayed on the granules from Spray 2 in a fluidized bed granulator under the conditions noted in Table 1. The bed temperature was maintained at 20° C. throughout the coater run.

TABLE 1
Running parameters:

| | Spray | Spray | Spray | |
|---|---|---|---|---|
| | 1 | 2 | 3 | |
| START RATE | 0.18 | 0.22 | 0.15 | Kg/min/nozzle |
| END RATE | 0.28 | 0.43 | 0.26 | Kg/min/nozzle |
| RAMP TIME | 90 | 30 | 60 | min. |
| SPEC. GRAVITY | 1.15 | 1.2 | 1.07 | |
| BED TEMP | 70 | 50 | 50 | ° C. |
| ATOM. AIR | 5.3 | 3.9 | 5.3 | BAR |
| PRES | | | | |

In the following examples, materials and conditions for the seed, Spray 1 and Spray 2 are identical to those in Example 1. Conditions for Spray 3 are substantially the same as those shown in Table 1.

### Example 2

Spray 3

Coating: 14.17% of Batch Weight

1. 2.50% Methylcellulose A-15

2. 2.50% Pure Cote B790

3. 6.00% TiO2

4. 1.50% Neodol 2.3-65 T

5. 1.67% PEG 600

This outer coating was batched as an 18% dry solids solution. Cold water was added to a vessel, then the Pure Cote B790 and TiO2 was added into the cold water. The Pure Cote and TiO2 was agitated for 10-15 minutes to aid in dispersion. After the Pure Cote and TiO2 has had time to disperse the temperature was brought up to 95° C. The temperature was kept at 95° C. for 30 minutes. While the temperature remained at 95° C., the methylcellulose (MC) A-15 was added. Generally, the MC disperses at a temperature above 60° C. After the 30 minutes at 95° C., the solution was cooled down to 20° C. At 30° C., the MC A-15 dissolved. The PEG 600 and Neodol were added at this time. After 30 minutes, this solution was used in the coater. The coating was sprayed on the granules from Spray 2 in a fluidized bed granulator under the conditions noted in Table 1. The bed temperature was maintained at 20° C. throughout the coater run.

### Example 3

Spray 3

Coating: 13.67% of Batch Weight

1. 1.25% Methylcellulose A-15

2. 3.75% Pure Cote B790

3. 6.00% TiO2

4. 1.00% Neodol 2.3-65 T

5. 1.67% PEG 600

This outer coating was batched as an 18% dry solids solution. Cold water was added to a vessel, then the Pure Cote B790 and TiO2 was added into the cold water. The Pure Cote and TiO2 was agitated for 10-15 minutes to aid in dispersion. After the Pure Cote and TiO2 has had time to disperse the temperature was brought up to 95° C. The temperature was kept at 95° C. for 30 minutes. While the temperature remained at 95° C., the methylcellulose (MC) A-15 was added. Generally, the MC disperses at a temperature above 60° C. After the 30 minutes at 95° C., the solution was cooled down to 20° C. At 30° C., the MC A-15 dissolved. The PEG 600 and Neodol were added at this time. After 30 minutes, this solution was used in the coater. The coating was sprayed on the granules from Spray 2 in a fluidized bed granulator under the conditions noted in Table 1. The bed temperature was maintained at 20° C. throughout the coater run.

### Example 4

Spray 3

Coating: 13.67% of Batch Weight

1. 2.50% Hydroxypropylmethylcellulose E-15

2. 2.50% Pure Cote B790

3. 6.00% TiO2

4. 1.00% Neodol 2.3-65 T

5. 1.67% PEG 600

This outer coating was batched as an 18% dry solids solution. Cold water was added to a vessel, then the Pure Cote B790 and TiO2 was added into the cold water. The Pure Cote and TiO2 was agitated for 10-15 minutes to aid in dispersion. After the Pure Cote and TiO2 has had time to disperse the temperature was brought up to 95° C. The temperature was kept at 95° C. for 30 minutes. While the temperature remained at 95° C., the hydroxypropyl methylcellulose (HPMC) E-15 was added. Generally, the HPMC disperses at a temperature above 60° C. After the 30 minutes at 95° C., the solution was cooled down to 20° C. At 30° C., the HPMC E-15 dissolved. The PEG 600 and Neodol were added at this time. After 30 minutes, this solution was used in the coater. The coating was sprayed on the granules from Spray 2 in a fluidized bed granulator under the conditions noted in Table 1. The bed temperature was maintained at 20° C. throughout the coater run.

### Example 5

Spray 3

Coating: 14.01 % of Batch Weight

1. 6.16% Pure Cote B790

2. 1.56% Glycerol

3. 6.00% TiO2

4. 0.29% Sodium Laurel Sulfate

This outer coating was batched as an 30% dry solids solution. Cold water was added to a vessel, then the Pure Cote B790 and TiO2 was added into the cold water. The Pure Cote and TiO2 was agitated for 10-15 minutes to aid in dispersion. After the Pure Cote and TiO2 has had time to disperse the temperature was brought up to 95° C. The temperature was kept at 95° C. for 30 minutes. While the temperature remained at 95° C., the glycerol and sodium laurel sulfate were added. After the 30 minutes at 95° C., the solution was cooled down to 20° C. The coating was sprayed on the granules from Spray 2 in a fluidized bed granulator under the conditions noted in Table 1. The bed temperature was maintained at 20° C. throughout the coater run.

### Example 6

Spray 3

Coating: 30% of Batch Weight

1. 14.94% Pure Cote B790

2. 4.20% Glycerol

3. 4.20% Carnauba Wax

4. 6.00% TiO2

5. 0.66% Sodium Laurel Sulfate

This outer coating was batched as an 30% dry solids solution. Cold water was added to a vessel, then the Pure Cote B790 and TiO2 was added into the cold water. The Pure Cote and TiO2 was agitated for 10-15 minutes to aid in dispersion. After the Pure Cote and TiO2 has had time to disperse the temperature was brought up to 95° C. The temperature was kept at 95° C. for 30 minutes. While the temperature remained at 95° C., the glycerol, carnauba wax and sodium laurel sulfate were added. After the 30 minutes at 95° C., the solution was cooled down to 20° C. The coating was sprayed on the granules from Spray 2 in a fluidized bed granulator under the conditions noted in Table 1. The bed temperature was maintained at 20° C. throughout the coater run.

Various other examples and modifications of the foregoing description and examples will be apparent to a person skilled in the art after reading the disclosure without departing from the spirit and scope of the invention, and it is intended that all such examples or modifications be included within the scope of the appended claims. All publications and patents referenced herein are hereby incorporated by reference in their entirety.

What is claimed is:

1. A coating comprising a modified starch and a plasticizer.

2. The coating of claim 1, wherein the coating further comprises a secondary polymer.

3. A coating comprising a modified starch and a secondary polymer.

4. A granule comprising a granule core and the coating of claim 1.

5. The granule of claim 4, wherein the granule core comprises an enzyme.

6. A granule comprising a granule core and the coating of claim 3.

7. The granule of claim 6, wherein the granule core comprises an enzyme.

8. A composition comprising a tablet and the coating of claim 1.

9. A composition comprising a tablet and the coating of claim 3.

10. A cleaning composition comprising the granule of claim 4.

11. A cleaning composition comprising the granule of claim 6.

12. The coating of claim 1, wherein the modified starch is a hydroxypropyl modified starch.

13. The coating of claim 3, wherein the modified starch is a hydroxypropyl modified starch.

14. A coated pharmaceutical dosage form comprising a pharmaceutical dosage form and the coating of claim 1.

15. A coated pharmaceutical dosage form comprising a pharmaceutical dosage form and the coating of claim 3.

16. A coated seed comprising a seed and the coating of claim 1.

17. A coated seed comprising a seed and the coating of claim 3.

18. A textile composition comprising the granule of claim 4.

19. A textile composition comprising the granule of claim 6.

20. A feed composition comprising the granule of claim 4.

21. A feed composition comprising the granule of claim 6.
